(19) United States
(12) Patent Application Publication
(10) Pub. No.: US 2026/0132118 A1
(43) Pub. Date: May 14, 2026

(54) SALT FORMS OF R-MDMA AND METHODS USING THE SAME

(71) Applicant: EmpathBio, Inc., New York, NY (US)

(72) Inventors: Majed FAWAZ, Foxborough, MA (US); Setu KASERA, Berlin (DE); Nicholas MORRA, Ontario (CA)

(21) Appl. No.: 19/431,012

(22) Filed: Dec. 23, 2025

Related U.S. Application Data
(63) Continuation of application No. 18/401,401, filed on Dec. 30, 2023.
(60) Provisional application No. 63/436,531, filed on Dec. 31, 2022.

(51) Int. Cl. C07D 317/58; A61K 31/36

(52) U.S. Cl. CPC C07D 317/58; A61K 31/36

(57) ABSTRACT

Pharmaceutical compositions including R-MDMA salts O-Methyl Mandelate Form A, Nitrate Form A, and Mandelate Form B exhibit properties that are favorable for a pharmaceutical active ingredient. Methods of treating disorders, including neurological disorders, by administration of these compositions are described.

### CROSS-REFERENCE TO RELATED APPLICATIONS

This application is a continuation of U.S. patent application Ser. No. 18/401,401, filed Dec. 30, 2023, which claims priority to and the benefit of U.S. Provisional Patent Application No. 63/436,531, filed Dec. 31, 2022, the content of each of which is incorporated herein by reference.

### FIELD OF THE INVENTION

The present disclosure relates to novel 3,4-Methylenedioxymethamphetamine (hereinafter “MDMA”) compositions and methods of treating neurological diseases and conditions. Specifically, the disclosure provides improved pharmaceutical compositions comprising MDMA in a salt form that exhibits favorable properties for a pharmaceutical active ingredient.

### BACKGROUND OF THE INVENTION

3,4-Methylenedioxymethamphetamine (MDMA) is a substituted amphetamine class, and its free base form is a colorless oil, insoluble in water. MDMA is known to exist as a racemate, in addition to its individual R and S enantiomers. MDMA free base exists as a non-crystalline oil or fluid. The most common salt of MDMA is a hydrochloride salt, which appears as a white or off-white powder or crystal and is soluble in water.

On the pharmacological level, MDMA is distinct from both LSD-type hallucinogens and amphetamines. MDMA itself is an amphetamine derivative that possesses complex pharmacology and is believed to act by increasing the release of monoamines like serotonin, norepinephrine and dopamine in the brain, as well as stimulating neurohormonal activity. These effects are thought to result in the anxiolytic, prosocial and empathic responses seen with MDMA administration in humans. As a result, MDMA is often classified as an entactogen rather than a typical psychedelic.

The prosocial effects of MDMA could play an important role in its therapeutic potential by increasing openness and facilitating a therapeutic alliance between patients and therapists. One of the most reliable behavioral features of MDMA is that it reduces aggression across species, along with reduced anxiety, lowered defensiveness, and a feeling of “closeness” with other persons. It is hypothesized that the effects of MDMA allows a person to voluntarily navigate difficult emotions when recalling traumatic memories due to a reduction in activity in the amygdala, a region of the brain activated during fear responses. This reduction in fear allows for a decreased defensiveness about revisiting painful experiences, making room for new perspectives and different responses towards deep-rooted traumatic memories.

Therefore, a significant need exists for readily administrable medications of MDMA to treat neurological diseases and conditions. Such medications, which maximize efficacy while enabling drug side effects to be effectively controlled, are of particular interest, especially if administrable via a convenient route, including self-administration. There remains a need for more stable compositions that are capable of providing the controlled release of MDMA.

### SUMMARY OF THE INVENTION

The present disclosure relates to salts of R-3,4-methylenedioxymethamphetamine

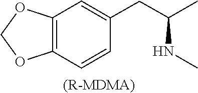

In certain aspects, the composition comprises a salt of R-3,4-Methylenedioxymethamphetamine (R-MDMA), wherein the salt is selected from a DL-Mandelate, Napsylate, Nitrate, O-methyl mandelate, or L-Tartarate salt of R-MDMA.

In embodiments, the present disclosure provides O-Methyl Mandelate Form A salt of R-MDMA.

In embodiments, O-Methyl Mandelate Form A is characterized by an X-ray Powder Diffraction (XPRD) pattern substantially similar to that shown in FIG. 3. In embodiments, O-Methyl Mandelate Form A is characterized by peaks in an XRPD pattern at 16.0±0.2, 18.2±0.2, 16.8±0.2, 23.0±0.2, 2.5±0.2, 16.2±0.2° 2θ. In embodiments, O-Methyl Mandelate Form A exhibits a monoclinic lattice wherein a=6.26±0.05 Å, b=51.09±0.05 Å, c=6.41±0.05 Å, α=90±1°, β=113.4±1°, γ=90±1°, and the cell volume is 1,882±5 3/cell.

In embodiments, O-Methyl Mandelate Form A exhibits a Differential Scanning Calorimetry (DSC) thermogram comprising an endotherm peak with an onset at 141±5° C. In embodiments, O-Methyl Mandelate exhibits a DSC thermogram comprising an endothermic peak at 142±5° C.

In embodiments, O-Methyl Mandelate Form A exhibits 0.7±0.1% weight loss between 42-211° C. as measured by thermogravimetric (TGA) analysis.

In embodiments, O-Methyl Mandelate Form A exhibits a melting point of 141±5° C.

In embodiments, O-Methyl Mandelate Form A exhibits 0.18±0.02% weight gain from 5-95% relative humidity (RH) and a 0.21±0.02% weight loss from 95-6% RH at 10% RH increments under a nitrogen purge as measured by Dynamic Vapor Sorption (DVS).

In embodiments, O-Methyl Mandelate Form A exhibits a solubility of 26±1 mg/mL.

In embodiments, O-Methyl Mandelate Form A exhibits physical stability upon stressing at 90% RH for 8 days.

In embodiments, the present disclosure provides Nitrate Form A salt of R-MDMA.

In embodiments, Nitrate Form A is characterized by an XRPD pattern substantially similar to that shown in FIG. 4. In embodiments, Nitrate Form A is characterized by peaks in an XRPD pattern at 15.8±0.2, 18.0±0.2, 13.5±0.2, 26.5±0.2, 23.2±0.2, 13.0±0.2°2θ.

In embodiments, Nitrate Form A exhibits an orthorhombic lattice wherein a=7.40±0.05 Å, b=9.82±0.05 Å, c=17.78±0.05 Å, a=90±1°, 3=90±1°, γ=90±1°, and the cell volume is 1,291±5 Å³/cell.

In embodiments, Nitrate Form A exhibits a Differential Scanning Calorimetry (DSC) thermogram comprising an endotherm peak with an onset at 135±5° C. In embodiments, Nitrate Form A exhibits a DSC thermogram comprising an endothermic peak at 136±5° C. In embodiments, Nitrate Form A exhibits a DSC thermogram comprising an exothermic peak at 153±5° C.

In embodiments, Nitrate Form A exhibits 0.1±0.11% weight loss between 52-136° C., 16.4±0.11% weight loss between 144-163° C., and 6.6±0.1% weight loss between 167-256° C. as measured by thermogravimetric (TGA) analysis.

In embodiments, Nitrate Form A exhibits a melting point of 135±5° C.

In embodiments, Nitrate Form A exhibits a solubility of 3±1 mg/mL.

In embodiments, Nitrate Form A exhibits physical stability upon stressing at 90% RH for 13 days.

In embodiments, the present disclosure provides Mandelate Form B salt of R-MDMA.

In embodiments, Mandelate Form B is characterized by an XRPD pattern substantially similar to that shown in FIG. 2. In embodiments, Mandelate Form B is characterized by peaks in an XRPD pattern at 19.8±0.2, 13.8±0.2, 17.0±0.2, 14.2±0.2, 18.5±0.2, 21.0±0.2°2θ.

In embodiments, Mandelate Form B exhibits a monoclinic lattice wherein a=9.02±0.05 Å, b=13.15±0.05 Å, c=15.69±0.05 Å, α=90±1° β=101±1°, γ=90±1°, and the cell volume is 1,823±5 Å³/cell.

In embodiments, Mandelate Form B exhibits a Differential Scanning Calorimetry (DSC) thermogram comprising an endotherm peak with an onset at 109±5° C. In embodiments, Mandelate Form B exhibits a DSC thermogram comprising an endothermic peak at 111±5° C.

In embodiments, Mandelate Form B exhibits 0.4±0.1% weight loss between 52-180° C. as measured by thermogravimetric (TGA) analysis.

In embodiments, Mandelate Form B exhibits a melting point of 109±5° C.

In embodiments, Mandelate Form B exhibits a solubility greater than 82 mg/mL.

In embodiments, Mandelate Form B exhibits physical stability upon stressing at 90% RH for 9 days.

In embodiments, the present disclosure provides pharmaceutical compositions comprising O-Methyl Mandelate Form A salt of R-MDMA. In embodiments, the pharmaceutical composition further comprises a pharmaceutically acceptable excipient. In embodiments, the present disclosure provides an oral dosage form comprising O-Methyl Mandelate Form A salt of R-MDMA.

In embodiments, the present disclosure provides pharmaceutical compositions comprising Nitrate Form A salt of R-MDMA. In embodiments, the pharmaceutical composition further comprises a pharmaceutically acceptable excipient. In embodiments, the present disclosure provides an oral dosage form comprising Nitrate Form A salt of R-MDMA.

In embodiments, the present disclosure provides pharmaceutical compositions comprising Mandelate Form B salt of R-MDMA. In embodiments, the pharmaceutical composition further comprises a pharmaceutically acceptable excipient. In embodiments, the present disclosure provides an oral dosage form comprising Mandelate Form B salt of R-MDMA.

The present disclosure provides methods of treating post-traumatic stress disorder (PTSD) in a subject in need thereof, comprising administering a therapeutically effective amount of a O-Methyl Mandelate Form A salt of R-MDMA, a Nitrate Form A salt of R-MDMA, a Mandelate Form B salt of R-MDMA a pharmaceutical composition, or an oral dosage form described herein to the subject.

The present disclosure provides methods of depression in a subject in need thereof, comprising administering a therapeutically effective amount of a O-Methyl Mandelate Form A salt of R-MDMA, a Nitrate Form A salt of R-MDMA, a Mandelate Form B salt of R-MDMA, a pharmaceutical composition, or an oral dosage form described herein to the subject. In embodiments, the depression is a major depressive disorder (MDD) or treatment-resistant depression (TRD).

The present disclosure provides methods of treating an anxiety disorder in a subject in need thereof, comprising administering a therapeutically effective amount of O-Methyl Mandelate Form A salt of R-MDMA, a Nitrate Form A salt of R-MDMA, a Mandelate Form B salt of R-MDMA, a pharmaceutical composition, or an oral dosage form to the subject.

### BRIEF DESCRIPTION OF THE FIGURES

FIG. 1 shows the overlay of the XPRD diffractograms of the obtained salts: Mandelate Material A, Mandelate Form B, O-Methyl Mandelate Form A, Napsylate Material A, Nitrate Form A, Tartaric Material A+tartaric acid, Tartaric Material B+Tartaric Material A+tartaric acid;

FIG. 2 shows the indexed XRPD diffractogram of a sample of R-MDMA Mandelate Form B;

FIG. 3 shows the indexed XRPD diffractogram of a sample of R-MDMA O-Methyl Mandelate;

FIG. 4 shows the indexed XRPD diffractogram of a sample of R-MDMA Nitrate From A;

FIG. 5 shows a ¹H-NMR spectrum for O-Methyl Mandelate Form A;

FIG. 6 shows a ¹H-NMR spectrum for Mandelate Material A;

FIG. 7 shows a ¹H-NMR spectrum for Mandelate Form B;

FIG. 8 shows a ¹H-NMR spectrum for Napsylate Material A;

FIG. 9 shows a ¹H-NMR spectrum for Nitrate Form A;

FIG. 10 shows a DSC thermogram (top) collected at a heating rate 10° C./minute and a TGA thermogram (bottom) of a sample of R-MDMA Mandelate Material A;

FIG. 11 shows a DSC thermogram (top) collected at a heating rate 10° C./minute and a TGA thermogram (bottom) of a sample of R-MDMA Mandelate Form B;

FIG. 12 shows a DSC thermogram (top) collected at a heating rate 10° C./minute and a TGA thermogram (bottom) of a sample of R-MDMA O-Methyl Mandelate Form A;

FIG. 13 shows a DSC thermogram (top) collected at a heating rate 10° C./minute and a TGA thermogram (bottom) of a sample of R-MDMA Napsylate Material A;

FIG. 14 shows a DSC thermogram (top) collected at a heating rate 10° C./minute and a TGA thermogram (bottom) of a sample of R-MDMA Nitrate Form A;

FIG. 15 shows a plot of melting points of the R-MDMA salts determined by DSC;

FIG. 16A shows a Dynamic Vapor Sorption (DVS) isotherm plot of R-MDMA O-Methyl Mandelate Form A; and

FIG. 16B shows a DVS isotherm analysis report of FIG. 16A.

### DETAILED DESCRIPTION

The present invention relates to salt compounds of R-MDMA and their polymorphs which are capable of stabilizing R-MDMA in a solid form to provide a controlled release of R-MDMA in a suitable dosage form. The R-MDMA salts O-Methyl Mandelate Form A, Nitrate Form A, and Mandelate Form B exhibit properties that are favorable for a pharmaceutical active ingredient. The invention also relates to methods of making these R-MDMA salt compositions, and methods of treatment of disease and disorders, e.g., neurological disorders such as post-traumatic stress disorder (PTSD), with the ability to promote neuroplasticity by administering these compositions to patients in need thereof.

Throughout this disclosure, various patents, patent applications and publications are referenced. The disclosures of these patents, patent applications and publications in their entireties are incorporated into this disclosure by reference for all purposes in order to describe the state of the art more fully as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The singular form “a”, “an” and “the” include plural references unless the context clearly dictates otherwise. For example, the term “a pharmaceutically acceptable carrier” may include a plurality of pharmaceutically acceptable carriers, including mixtures thereof.

The term “and/or” is intended to mean either or both of two components of the invention.

The term “subject,” “individual” and “patient” are used interchangeably herein and refers to a human.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “preventing” as used herein with regard to a patient or subject, refers to preventing the onset of disease development if none had occurred, preventing the disease or disorder from occurring in a subject or a patient that may be predisposed to the disorder or disease but has not yet been diagnosed as having the disorder or disease, and/or preventing further disease/disorder development if already present.

The term “substantially similar” as used herein with regards to an analytical spectrum, such as an XRPD pattern, means that a spectrum resembles the reference spectrum in both the peak locations and their relative intensities, allowing for variability appropriate in the art. For example, two spectra may be regarded as “substantially similar” when the two spectra share defining characteristics sufficient to differentiate them from a spectrum obtained for a different solid form. In embodiments, spectra or characterization data that are substantially similar to those of a reference crystalline form are understood by those of ordinary skill in the art to correspond to the same crystalline form as the particular reference. In analyzing whether spectra or characterization data are substantially similar, a person of ordinary skill in the art understands that particular characterization data points may vary to a reasonable extent while still describing a given solid form, due to, for example, experimental error and routine sample-to-sample analysis.

The term “treating” as used herein with regard to a patient or subject, refers to improving at least one symptom of the patient's or subject's disorder. In some embodiments, treating can be improving, or at least partially ameliorating a disorder or one or more symptoms of a disorder.

The term “in need of treatment” and the term “in need thereof” when referring to treatment are used interchangeably and refer to a judgment made by a caregiver (e.g. physician, nurse, nurse practitioner) that a patient will benefit from treatment.

The terms “treat” and “treatment” refer herein to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition. “Treatment” can also mean prolonging survival as compared to expected survival if not receiving treatment. Those in need of treatment include those already with the disease or condition as well as those prone to having the disease or condition or those in which the disease or condition is to be prevented. “Treatment” can, when concerning depression, also include reducing at least one sign or symptom of depression. Examples of a sign or symptom of depression include depressed mood, diminished interest in activities, weight loss or gain, decrease or increase in appetite, insomnia or hypersomnia, psychomotor agitation or retardation, fatigue or loss of energy, feelings of worthlessness or excessive or inappropriate guilt, diminished ability to concentrate or indecisiveness, or suicidal ideation or behavior.

The term “pharmaceutically acceptable” as used herein, refers to a component of a pharmaceutical composition that is compatible with the other ingredients of the formulation and not overly deleterious to the recipient thereof.

The term “effective amount” or “therapeutically effective amount” as used herein, refers to the amount of active agent that elicits the biological or medicinal response in a tissue, system, or individual that is being sought by a researcher, healthcare provider or individual.

The term “neurological disease or condition” as used herein, means a disease or condition selected from: a neuropsychiatric disorder, such as depression (including severe depression such as treatment-resistant depression, major depressive disorder and persistent depressive disorder), catatonic depression, a depressive disorder due to a medical condition, postpartum depression, premenstrual dysphoric disorder, or seasonal affective disorder, anxiety, anxiety disorder, social anxiety disorder, general anxiety disorder (GAD), avolition disorder, bipolar disorder (including bipolar I disorder and bipolar II disorder), post-traumatic stress disorder, body dysmorphic disorder, abnormalities of mood or emotion, including the above conditions, dysthymia, schizoaffective disorder, schizophrenia and other psychotic disorders, panic disorder, traumatic stress disorders, phobic disorders, and personality disorders with abnormal mood, such as borderline personality disorder, schizoid and schizotypal disorders and suicide ideation, or rumination/unproductive repetitive thoughts negatively impacting one's behavior/mood/ability to focus, obsessive-compulsive disorder, addiction (including substance use disorder such as addiction to nicotine, alcohol, cocaine, opioids, amphetamine, methamphetamine, heroin, morphine, phencyclidine, 3,4-methylenedioxy-methamphetamine, as well as other addictive substances), addictive behavior (including eating, gambling, sex, pornography, videogames, work, exercise, spiritual obsession, self-harm, travel and shopping addiction), eating disorder (including anorexia nervosa, bulimia nervosa and binge eating disorder), and pain (including pain associated with migraine or headache or chronic pain).

The term “R-3,4-methylenedioxymethamphetamine” or “R-MDMA”, includes the compound of formula (I):

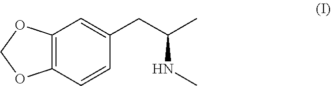

including pharmaceutically acceptable forms of R-MDMA. The term “R-MDMA free form” or “R-MDMA free base” refers to the compound of formula (I) without a pharmaceutically acceptable salt. In another aspect, the compound of formula (I) as a base may be combined with one or more acidic moieties of formula (II)-(X).

The term “4-aminosalicyclic acid”, includes an acidic moiety of formula (II):

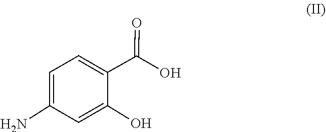 [SMILES: Nc1ccc(C(=O)O)c(O)c1]

The term “aspartic acid”, includes an acidic moiety of formula (III):

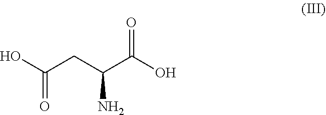

The term “benzoic acid”, includes an acidic moiety of formula (IV):

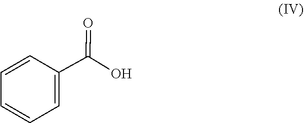

The term “mandelic acid”, includes an acidic moiety of formula (V):

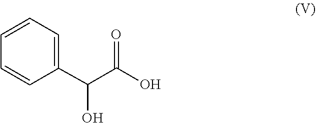 [SMILES: O=C(O)C(O)c1ccccc1]

The term “naphthalene-2-sulfonic acid” or “napsylic”, includes an acidic moiety of formula (VI):

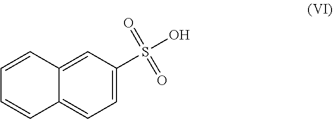 [SMILES: O=S(=O)(O)c1ccc2ccccc2c1]

The term “nitric acid”, includes an acidic moiety of formula (VII):

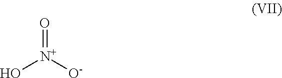

The term “o-methyl mandelic acid”, includes an acidic moiety of formula (VIII):

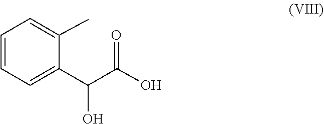

The term “succinic acid”, includes an acidic moiety of formula (IX):

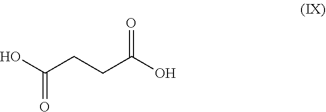

The term “tartaric acid”, includes an acidic moiety of formula (X):

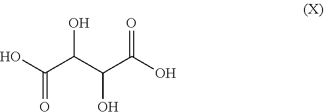

In certain preferred embodiments, the present inventors have found that forms of the O-methyl mandelate salt, the mandelate salt, and the nitrate salt of R-MDMA are particularly suitable for pharmaceutical applications.

All XRPD peaks and patterns are given in °2θ using Cu Kal radiation at a wavelength of 1.5406 Å. The values of degree 2θ allow appropriate error margins. For example, the degree 2θ of about “17.48±0.2” denotes a range from about 17.46 to 17.50 degree 2θ. Depending on the sample preparation techniques, the calibration techniques applied to the instruments, human operational variation, and etc., those skilled in the art recognize that the appropriate error of margins for a XRPD can be ±0.2, which includes any value below ±0.2 such as +0.1; +0.05; or less.

TGA and DSC thermograms for a given crystalline form of the same compound will vary within a margin of error. The values of a single peak, expressed in degree Celsius, allow appropriate error margins. Typically, the error margins are represented by “±”. For example, the single peak characteristic value of about “120±5” denotes a range from about 115 to 125. Depending on the sample preparation techniques, the calibration techniques applied to the instruments, human operational variations, and etc., those skilled in the art recognize that the appropriate error of margins for a single peak characteristic value can be ±5, which includes any value below ±5 such as ±4, ±3.5, ±3, ±2.5; ±2.0; ±1.5; ±1.0; ±0.5; or less.

The following description includes information that may be useful in understanding the present invention. It is not an admission that any of the information provided herein is prior art or relevant to the presently claimed inventions, or that any publication specifically or implicitly referenced is prior art.

### Salt Forms of R-3,4-methylenedioxymethamphetamine (R-MDMA)

In one aspect, the present disclosure provides salt forms of R-MDMA. In embodiments, the salt form of R-MDMA is salt O-Methyl Mandelate Form A. In embodiments, the salt form of R-MDMA is salt Nitrate Form A. In embodiments, the salt form of R-MDMA is salt Mandelate Form B.

### Mandelate Form B Salt of R-MDMA

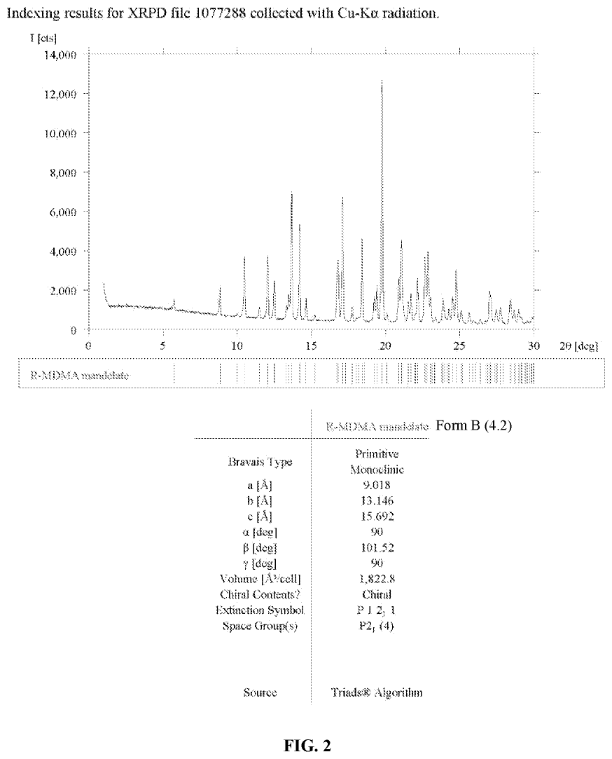

In embodiments, Mandelate Form B is characterized by an XRPD pattern substantially similar to that shown in FIG. 2. In embodiments, Mandelate Form B is characterized by peaks in an XRPD pattern at 19.8±0.2, 13.8±0.2, 17.0±0.2, 14.2±0.2, 18.5±0.2, 21.0±0.2 020. In embodiments, Mandelate Form B exhibits a primitive monoclinic lattice wherein a=9.02±0.05 Å, b=13.15±0.05 Å, c=15.69±0.05 Å, α=90±1°, β=101±1°, γ=90±1°, and the cell volume is 1,823±5 Å³/cell.

In embodiments, Mandelate Form B exhibits a Differential Scanning Calorimetry (DSC) thermogram comprising an endotherm peak with an onset at 109±5° C. In embodiments, Mandelate Form B exhibits a DSC thermogram comprising an endothermic peak at 111±5° C.

In embodiments, Mandelate Form B exhibits 0.4±0.1% weight loss between 52-180° C. as measured by thermogravimetric (TGA) analysis.

In embodiments, Mandelate Form B exhibits a melting point of 109±5° C.

In embodiments, Mandelate Form B exhibits a solubility greater than 82 mg/mL.

In embodiments, Mandelate Form B exhibits physical stability upon stressing at 90% RH for 9 days.

### O-Methyl Mandelate Form Salt of R-MDMA

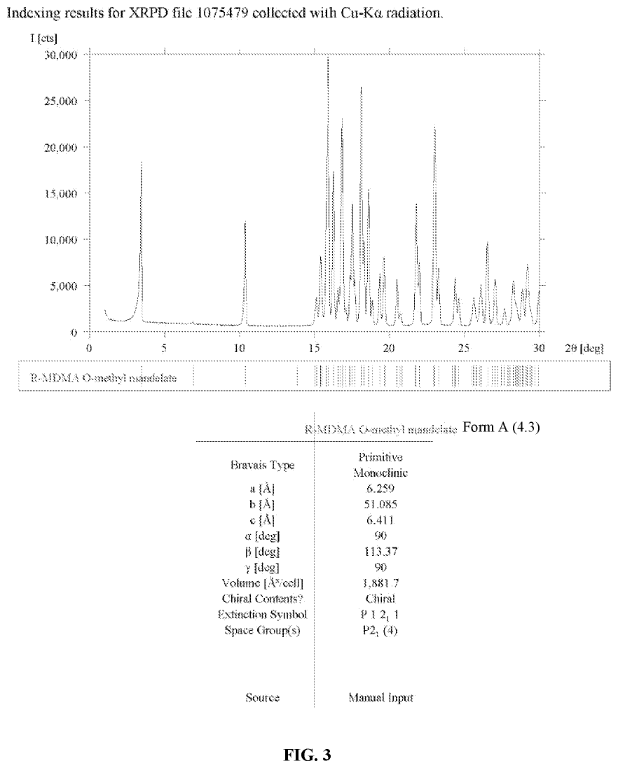

In embodiments, O-Methyl Mandelate Form A is characterized by an XRPD pattern substantially similar to that shown in FIG. 3. In embodiment, O-Methyl Mandelate Form A is characterized by peaks in an XRPD pattern at 16.0±0.2, 18.2±0.2, 16.8±0.2, 23.0±0.2, 2.5±0.2, 16.2±0.2°2θ. In embodiments, O-Methyl Mandelate Form A exhibits a primitive monoclinic lattice wherein a=6.26±0.05 Å, b=51.09±0.05 Å, c=6.41±0.05 Å, α=90±1°, β=113.4±1°, γ=90±1°, and the cell volume is 1,882±5 Å³/cell.

In embodiments, O-Methyl Mandelate Form A exhibits a Differential Scanning Calorimetry (DSC) thermogram comprising an endotherm peak with an onset at 141±5° C. In embodiments, O-Methyl Mandelate exhibits a DSC thermogram comprising an endothermic peak at 142±5° C.

In embodiments, O-Methyl Mandelate Form A exhibits 0.7±0.1% weight loss between 42-211° C. as measured by thermogravimetric (TGA) analysis.

In embodiments, O-Methyl Mandelate Form A exhibits a melting point of 141±5° C.

In embodiments, O-Methyl Mandelate Form A exhibits 0.18±0.02% weight gain from 5-95% relative humidity (RH) and a 0.21±0.02% weight loss from 95-6% RH at 10% RH increments under a nitrogen purge as measured by Dynamic Vapor Sorption (DVS).

In embodiments, O-Methyl Mandelate Form A exhibits a solubility of 26±1 mg/mL.

In embodiments, O-Methyl Mandelate Form A exhibits physical stability upon stressing at 90% RH for 8 days.

### Nitrate Form A Salt of R-MDMA

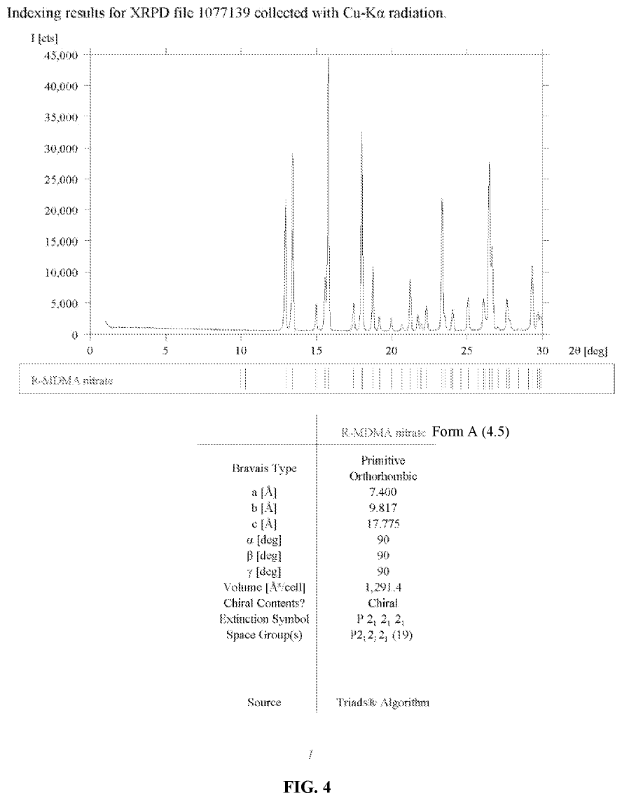

In embodiments, Nitrate Form A is characterized by an XRPD pattern substantially similar to that shown in FIG. 4. In embodiments, Nitrate Form A is characterized by peaks in an XRPD pattern at 15.8±0.2, 18.0±0.2, 13.5±0.2, 26.5±0.2, 23.2±0.2, 13.0±0.2°2θ. In embodiments, Nitrate Form A exhibits a primitive orthorhombic lattice wherein a=7.40±0.05 Å, b=9.82±0.05 Å, c=17.78±0.05 Å, α=90±1°, β=90±1°, γ=90±1°, and the cell volume is 1,291±5 Å³/cell.

In embodiments, Nitrate Form A exhibits a Differential Scanning Calorimetry (DSC) thermogram comprising an endotherm peak with an onset at 135±5° C. In embodiments, Nitrate Form A exhibits a DSC thermogram comprising an endothermic peak at 136±5° C. In embodiments, Nitrate Form A exhibits a DSC thermogram comprising an exothermic peak at 153±5° C.

In embodiments, Nitrate Form A exhibits 0.1±0.11% weight loss between 52-136° C., 16.4±0.11% weight loss between 144-163° C., and 6.6±0.1% weight loss between 167-256° C. as measured by thermogravimetric (TGA) analysis.

In embodiments, Nitrate Form A exhibits a melting point of 135±5° C.

In embodiments, Nitrate Form A exhibits a solubility of 3±1 mg/mL.

In embodiments, Nitrate Form A exhibits physical stability upon stressing at 90% RH for 13 days.

In embodiments, the present disclosure provides Mandelate Form B salt of R-MDMA.

### Pharmaceutical Compositions

In one aspect, the present disclosure provides compositions comprising at least one salt form of R-MDMA described herein (e.g., O-Methyl Mandelate Form A, Nitrate Form A, Mandelate Material A and/or Mandelate Form B) and one or more excipients. In embodiments, the composition is a pharmaceutical composition comprising a salt form of R-MDMA, and one or more pharmaceutically acceptable excipients.

In embodiments, the pharmaceutical composition comprises O-Methyl Mandelate Form A salt of R-MDMA.

In embodiments, the pharmaceutical composition comprises Nitrate Form A salt of R-MDMA.

In embodiments, the pharmaceutical composition comprises Mandelate Material A salt of R-MDMA.

In embodiments, the pharmaceutical composition comprises Mandelate Form B salt of R-MDMA.

In embodiments, the composition comprises a pharmaceutically acceptable carrier. In embodiments, a pharmaceutically acceptable carrier includes a pharmaceutically acceptable excipient, binder, adjuvant, and/or diluent. In embodiments, suitable pharmaceutically acceptable carriers include, but are not limited to, inert solid fillers or diluents and sterile aqueous or organic solutions. In embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2θ05).

In embodiments, one or more salt form of R-MDMA described herein (e.g., O-Methyl Mandelate Form A, Nitrate Form A, and/or Mandelate Form B) of the present disclosure are formulated for administration by a variety of means including orally, parenterally, by inhalation spray, topically, or rectally in formulations containing pharmaceutically acceptable carriers, adjuvants, vehicles, or mixtures thereof. The term parenteral as used here includes subcutaneous, intravenous, intramuscular, and intraarterial injections with a variety of infusion techniques. Intraarterial and intravenous injection as used herein includes administration through catheters.

In embodiments, the present disclosure provides oral dosage forms comprising a composition described herein.

In embodiments, the oral dosage form is a solid dosage form such as a tablet, capsule, pill, powder, or granule. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art.

### Therapeutic Methods

In one aspect, the present disclosure provides methods of treating or preventing neurological disorders in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject.

In embodiments, the neurological disorder is a mood disorder. In embodiments, the mood disorder is clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, major depressive disorder, minor depressive disorder, bipolar disorder or manic depressive disorder, depression caused by chronic medical conditions, treatment-resistant depression, refractory depression, suicidality, suicidal ideation, or suicidal behavior. In embodiments, the method described herein provides therapeutic effect to a subject suffering from depression (e.g., moderate or severe depression). In embodiments, the mood disorder is associated with neuroendocrine diseases and disorders, neurodegenerative diseases and disorders (e.g., epilepsy), movement disorders, tremor (e.g., Parkinson's Disease), or women's health disorders or conditions. In embodiments, the mood disorder is depression. In embodiments, the mood disorder is treatment-resistant depression or major depressive disorder. In embodiments, the mood disorder is major depressive disorder. In embodiments, the mood disorder is treatment-resistant depression.

In embodiments, the present disclosure provides methods of treating or preventing PTSD, mood disorders, general anxiety disorder, addictive disorders, and/or drug dependence in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject.

In embodiments, the present disclosure provides methods of treating or preventing PTSD in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject.

In embodiments, the present disclosure provides methods of treating or preventing behavioral or mood disorders in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject. In embodiments, the behavioral or mood disorder includes anxiety, such as social anxiety in autistic subjects (e.g., autistic adults) and anxiety related to life-threatening illnesses. In embodiments, the behavioral or mood disorder includes stress (where moderation thereof is measured, e.g., by effects on amygdala responses). In embodiments, the anxiety disorder is panic disorder, obsessive-compulsive disorder, and/or general anxiety disorder. In embodiments, the subject suffers from a lack of motivation, attention, lack of accuracy in memory recall, speed of response, perseveration, and/or cognitive engagement. Further examples include depression (e.g., MDD or TRD), attention disorders, disorders of executive function and/or cognitive engagement, obsessive compulsive disorder, bipolar disorder, panic disorder, phobia, schizophrenia, psychopathy, antisocial personality disorder and/or neurocognitive disorders.

In embodiments, the present disclosure provides methods for treating or preventing an addictive disorder in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject. In embodiments, the addictive disorder is alcohol abuse, substance abuse, smoking, obesity, or mixtures thereof. In embodiments, the disorder is an eating disorder (e.g., anorexia nervosa, bulimia, nervosa, binge eating disorder, etc.) or an auditory disorder.

In embodiments, the present disclosure provides methods for treating or preventing an impulsive disorder in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject. In embodiments, the impulsive disorder is attention deficit hyperactivity disorder (ADHD), attention deficit disorder (ADD), Tourette's syndrome, autism, or combinations thereof.

In embodiments, the present disclosure provides methods for treating or preventing a compulsive disorder in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject. In embodiments, the compulsive disorder is obsessive compulsive disorder (OCD), gambling, aberrant sexual behavior, or combinations thereof.

In embodiments, the present disclosure provides methods for treating or preventing a personality disorder in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject. In embodiments, the personality disorder is conduct disorder, antisocial personality, aggressive behavior, or combinations thereof to the subject.

In embodiments, the present disclosure provides methods of treating or preventing PTSD, social anxiety disorder (e.g., social anxiety in autism spectrum disorder), autism spectrum disorder, binge eating disorder, alcohol use disorder, treatment resistant depression, major depressive disorder, generalized anxiety disorder, schizophrenia, borderline personality disorder, opioid use disorder, narcissistic personality disorder, avoidant personality disorder, tinnitus, anorexia nervosa, substance use disorder, chronic pain, tobacco addiction, bulimia nervosa, antisocial personality disorder, ADHD, a traumatic brain injury, body dysmorphia, hypoactive sexual desire disorder, migraines, agoraphobia, narcolepsy, obsessive compulsive disorder, and/or fibromyalgia in a subject in need thereof, the methods comprising administering a therapeutically effective amount of a salt form of R-MDMA described herein (e.g., O-Methyl Mandelate Form A, Nitrate Form A, and/or Mandelate Form B) or a pharmaceutical composition thereof to the subject.

### Examples

The following example details the preparation and isolation of salt forms of R-MDMA (e.g., O-Methyl Mandelate Form A, Nitrate Form A, and Mandelate Form B). The study shows that O-Methyl Mandelate Form A exhibits properties that are favorable for a pharmaceutical active ingredient. O-Methyl Mandelate Form A consists of an anhydrous/unsolvated mono-salt confirmed to exist as a single phase with a melt onset at 141° C., approximate aqueous solubility of 26 mg/mL, low kinetic hygroscopicity, and physical stability at 90% RH. In particular, the kinetic hygroscopicity of O-Methyl Mandelate Form A is significantly lower than that of HCl Form I (0.2% sorption/desorption versus 5.3% sorption/desorption, respectively, between 5% and 95% RH).

### Screening Experiments

Screening experiments were performed to identify stable and polymorphic salt forms of R-MDMA. Twenty-three salt screen experiments were conducted with 9 different acids as seen in Table 1 below. Seven confirmed or suspected crystalline salts were produced from five acids listed in Table 2-6 below. The salt candidates were evaluated by XRPD, ¹H NMR, DSC, TGA, RH stressing, and aqueous solubility estimation.

TABLE 1

| | Acid | pKa | MW |
|---|---|---|---|
| 1 | 4-Aminosalicylic acid | 3.25, 10, 3.5 | 153.14 |
| 2 | Aspartic acid | 1.88 | 133.10 |
| 3 | Benzoic acid | 4.19 | 122.12 |
| 4 | DL-Mandelic acid | 3.37 | 152.15 |
| 5 | Napsylic acid | 0.17 | 208.24 |
| | (napthalene-2-sulfonic | | |
| | acid) | | |
| 6 | Nitric acid | −1.32 | 63.01 |
| 7 | O-methyl mandelic acid | 3.41 | 166.17 |
| 8 | Succinic acid | 4.21, 5.64 | 118.09 |
| 9 | L-Tartaric acid | 3.02, 4.36 | 150.09 |

Salt crystallization attempts involved the combination of R-MDMA free base with one molar equivalent of an acid in a given solvent. R-MDMA free base was obtained either as an as-received oil (Tables 4, 5, and 6) or a bulk solution in acetone resulting from the breaking of a R-MDMA HCl salt (Table 2-6). Additionally, solutions from unsuccessful salt attempts were evaporated to dryness and stirred in a different solvent to explore multiple crystallization conditions as seen in Table 3 and 5. Acetone, ethyl acetate, or acetonitrile were utilized for most experiments. The combination of the acid and R-MDMA free base typically resulted in a clear solution or slurry. The resulting mixtures/solutions were stirred for a stated duration. Any precipitated solids were collected and analyzed. For samples that did not exhibit precipitation at room temperature, additional techniques such as cooling and/or addition of an antisolvent (e.g., heptane or diethyl ether) were employed in an effort to induce precipitation of a crystalline salt.

In an exemplary embodiment, O-Methyl Mandelate Form A was prepared according to the conditions in Table 2 below.

TABLE 2
Salt attempts using O-Methyl Mandelic acid

| API/acid m/m | Conditions | Observations | XRPD Result |
|---|---|---|---|
| 0.7M R-MDMA in | 1) Add acetone to API w/ | 1) — | O-Methyl Mandelate |
| acetone 1:1 | stirring | 2) Cloudy white slurry | Form A |
| | 2) Add acid w/ stirring | 3) White/cream solids, fines, B/E | |
| | 3) Stir at room temp., 1 day | 4) White solids | |
| | 4) Filter | | |

In the salt attempt using O-Methyl Mandelic acid, the active pharmaceutical ingredient (API) 0.7 M R-MDMA in acetone was mixed with 1:1 molar equivalent of O-Methyl Mandelic acid while stirring. A cloudy white slurry formed upon mixing of the O-Methyl Mandelic acid and R-MDMA. After a day of mixing at room temperature, white solids were filtered and identified at O-Methyl Mandelate Form A via XRPD.

In an exemplary embodiment, Nitrate Form A was prepared according to the conditions in Table 3 below.

TABLE 3
Salt attempts using Nitric acid

| API/acid m/m | Conditions | Observations | XRPD Result |
|---|---|---|---|
| 0.7M R-MDMA in | 1) Add acetone to API w/ | 1) — | — |
| acetone 1:1 | stirring | 2) Clear slight yellow soln. | |
| | 2) Add acid w/ stirring | 3) Clear soln. | |
| | 3) Stir at room temp., 2 days | 4) Clear slightly yellow soln. | |
| | 4) Stir in freezer, 4 days | 5) Cloudy soln. w/ brown oil | |
| | 5) Add 2 mL Et₂O | 6) Slightly cloudy soln. w/ | |
| | 6) Stir in freezer, 8 days | light brown oil | |
| | 7) FE at room temp., 35 days | 7) Clear brown oil | |
| oil from previous | 1) Add EtOAc to oil w/ | 1) White cloudy soln. | Nitrate Form A |
| salt attempt | stirring | 2) Clear soln. white fines & | |
| 1:1 | 2) Stir at room temp, 1 day | aggregates, B/E | |
| | 3) Filter | 3) — | |

In the salt attempt using Nitric Acid, the API 0.7 M R-MDMA in acetone was mixed with 1:1 molar equivalent of Nitric acid while stirring to form a slight yellow solution. The solution was stirred for two days at room temperature to form a clear solution. The solution was then cooled in a freezer and stirred for 4 days to form a slight yellow solution. 2 mL Et₂O was added to the solution to form a cloudy solution with brown oil. The solution was then stirred in the freezer for 8 days to form a slightly cloudy solution. The solution freely evaporated at room temperature for 35 days to form a clear brown oil. The oil from this salt attempt was used for second attempt. Ethyl acetate was added to the oil while stirring to form a white cloudy solution. The solution was stirred at room temperature for a day, filtered, and identified as Nitrate Form A via XRPD.

In an exemplary embodiment, Mandelate Material A and Mandelate Form B was prepared according to the conditions in Table 4 below.

TABLE 4
Salt attempts using Mandelic acid

| API/acid m/m | Conditions | Observations | XRPD Result |
|---|---|---|---|
| (0.7M R-MDMA in | 1) Add acetone to API w/ | 1) — | Mandelate |
| acetone) 1:1 | stirring | 2) Clear soln. | Material A |
| | 2) Add acid w/ stirring | 3) White small needles | |
| | 3) Stir at room temp., 2 days | and prisms, B/E | |
| | 4) Filter | 4) — | |
| R-MDMA Free | 1) Add API and acid to vial | 1) — | Mandelate |
| Base oil | 2) Add EtOAc w/ stirring | 2) Clear soln. | Form B |
| 1:1 | 3) Stir at room temp., 3 days | 3) White fines and | |
| | 4) Filter | aggregates, B/E | |
| | | 4) White solids | |

In the salt attempt using Mandelic acid, the API 0.7 M R-MDMA in acetone was mixed with 1:1 molar equivalent of Mandelic acid while stirring to form clear solution. The solution was stirred at room temperature for 2 days and filtered. The product was identified as Mandelate Material Form A via XRPD. In a second attempt, R-MDMA free base oil was mixed with 1:1 molar equivalent of Mandelic acid. Ethyl acetate was added to the solution and stirred at room temperature for 3 days. White solids were filtered and identified at Mandelate Form B via XRPD.

In an exemplary embodiment, Napsylate Material A was prepared according to the conditions in Table 5 below.

TABLE 5
Salt attempts using Napsylic acid

| API/acid m/m | Conditions | Observations | XRPD Result |
|---|---|---|---|
| 0.7M R-MDMA in | 1) Add acetone to API | 1) — | — |
| acetone 1:1 | w/ stirring | 2) Cloudy brown soln. | |
| | 2) Add acid w/ stirring | 3) Clear yellow soln. | |
| | 3) Stir at room temp., 2 days | 4) Slight brown oily soln. | |
| | 4) Stir in freezer, 4 days | 5) Cloudy soln. w/ brown | |
| | 5) Add 1 mL Et₂O | oil | |
| | 6) Stir in freezer, 8 days | 6) Slightly cloudy soln. | |
| | 7) FE at room temp., 35 days | w/ light brown oil | |
| | | 7) Brown oil w/ B/E flakes | |
| oil from previous | 1) Add EtOAc to oil w/ | 1) Cloudy brown soln. | Napsylate |
| salt attempt 1:1 | stirring | 2) Brown clear soln. | Material A |
| | 2) Stir at room temp., 1 day | white fines & aggregates, | |
| | 3) Filter | B/E | |
| | | 3) — | |
| R-MDMA free base | 1) Add API and acid to vial | 1) — | — |
| oil 1:1 | 2) Add acetonitrile w/ stirring | 2) Clear soln. w/ brown | |
| | 3) Stir at room temp., 3 days | clumps | |
| | 4) Stir in freezer, 8 days | 3) Clear brown soln. | |
| | 5) S/AS w/ heptane | 4) Clear brown soln. | |
| | 6) Stir in freezer, 4 days | 5) Clear brown soln. | |
| | | 6) Bilayer soln. clear upper | |
| | | layer, brown lower layer | |

In an exemplary embodiment, Tartaric Material A and Tartaric Material B+A was prepared according to the conditions in Table 6 below.

TABLE 6
Salt attempts using Tartaric acid

| API/acid m/m | Conditions | Observations | XRPD Result |
|---|---|---|---|
| 0.7M R-MDMA in | 1) Added acetone to API | 1) — | Tartaric Material A + |
| acetone 1:1 | w/ stirring | 2) Suspended white solids | tartaric acid |
| | 2) Add acid w/ stirring | 3) White agglomerates, | |
| | 3) Stir at room temp., | some B/E | |
| | 2 days | | |
| R-MDMA Free | 1) Add API and acid | 1) — | Tartaric Material B |
| base oil 1:0.5 | 2) Add API w/ stirring | 2) Clear soln. w/ | B + A + tartaric acid |
| | 3) Stir at room temp., | 3) Small white needles, B/E | |
| | 3 days | 4) White solids | |
| | 4) Filter | | |

### Characterization of Salt Screen Products

The solids resulting from the various salt preparation attempts were qualitatively evaluated for crystallinity by XRPD. XRPD indexing was attempted for new crystalline patterns.

### X-Ray Powder Diffraction (XPRD) Experiments

XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5. The data acquisition parameters for each pattern are displayed above the image in the Data section of this report. All images have the instrument labeled as X'Pert PRO MPD regardless of the instrument used.

Within each figure referenced for a given indexed XRPD pattern (FIG. 2, 3, 4), agreement between the allowed peak positions, marked with bars, and the observed peaks indicates a consistent unit cell determination. Successful indexing of a pattern indicates that the sample is composed primarily of a single crystalline phase unless otherwise stated. Space groups consistent with the assigned extinction symbol, unit cell parameters, and derived quantities are tabulated below the respective figure. To confirm the tentative indexing solution, the molecular packing motifs within the crystallographic unit cells must be determined. No attempts at molecular packing were performed.

### Confirmed or Suspected Salts:

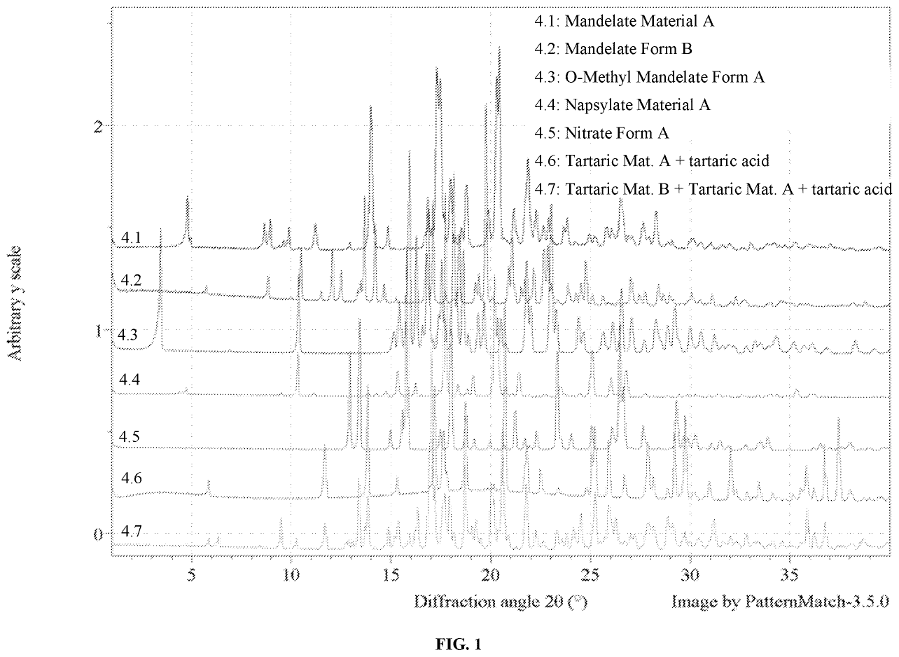

Experiments with 4-aminosalicylic, DL-aspartic, benzoic, and succinic acids were unsuccessful in producing crystalline salts. Seven confirmed or suspected salts produced from salt screening experiments, shown in Table 7, include the following: Mandelate Material A, Mandelate Form B, O-Methyl Mandelate Form A, Napsylate Material A, Nitrate Form A, Tartaric Material A+tartaric acid, and Tartaric Material B+Tartaric Material A+tartaric acid. An overlay of the XRPD of the obtained salts are shown in FIG. 1. The two potential tartrate materials were observed only in mixtures with unreacted tartaric acid and were not characterized further. The XRPD indexing results for the respective salts are shown in Table 7 below.

TABLE 7

| Salt | Indexed |
|---|---|
| Mandelate Material A | No |
| Mandelate Form B | Yes—fits unsolvated mono-salt |
| O-Methyl Mandelate Form A | Yes—fits unsolvated mono-salt |
| Napsylate Material A | No |
| Nitrate Form A | Yes—fits unsolvated mono-salt |
| Tartaric Material A + tartaric acid | No—mixtures |
| Tartaric Material B + Tartaric | No—mixtures |
| Material A + tartaric acid | |

### Mandelate Form B:

The XRPD pattern of Mandelate Form B was indexed and is shown in FIG. 2. Observed diffraction peaks are shown in FIG. 2. Crystal data of Mandelate Form B is shown in Table 8 below.

TABLE 8

| Crystal data | Mandelate Form B |
|---|---|
| Crystal system | Monoclinic |
| Space group | P2₁ (4) |
| Cell parameters | a = 9.018 Å |
| | b = 13.146 Å |
| | c = 15.692 Å |
| | α = 90° |
| | β = 101.52° |
| | γ = 90° |
| Unit cell volume | 1,822.8 |
| (Å3) | |

### O-Methyl Mandelate Form A:

The XRPD pattern of O-Methyl Mandelate Form A was indexed and is shown in FIG. 3. Observed diffraction peaks are shown in FIG. 3. Crystal data of O-Methyl Mandelate Form A is shown in Table 9 below.

TABLE 9

| | O-Methyl Mandelate |
|---|---|
| Crystal data | Form A |
| Crystal system | Monoclinic |
| Space group | P2₁ (4) |
| Cell parameters | a = 6.259 Å |
| | b = 51.085 Å |
| | c = 6.411 Å |
| | α = 90° |
| | β = 113.37° |
| | γ = 90° |
| Unit cell volume | 1,881.7 |
| (Å3) | |

### Nitrate Form A:

The XRPD pattern Nitrate Form A was indexed and is shown in FIG. 4. Observed diffraction peaks are shown in FIG. 4. Crystal data of Nitrate Form A is shown in Table 10 below.

TABLE 10

| Crystal data | Nitrate Form A |
|---|---|
| Crystal system | Orthorhombic |
| Space group | P2₁2₁2₁ (19) |
| Cell parameters | a = 7.400 Å |
| | b = 9.817 Å |
| | c = 17.775 Å |
| | α = 90° |
| | β = 90° |
| | γ = 90° |
| Unit cell volume | 1,291.4 |
| (Å3) | |

### Solution ¹H NMR Experiments

Solution proton NMR spectroscopy was used to confirm composition and stoichiometry, verify that chemical degradation did not occur, and evaluate the amount of solvent present in the remaining five salts.

The solution NMR spectra were acquired with an Avance 600 MHz spectrometer. The samples were prepared by dissolving approximately 5-10 mg of sample in DMSO-d6 containing TMS. The data acquisition parameters are presented on the ¹H-NMR spectra provided in the following figures.

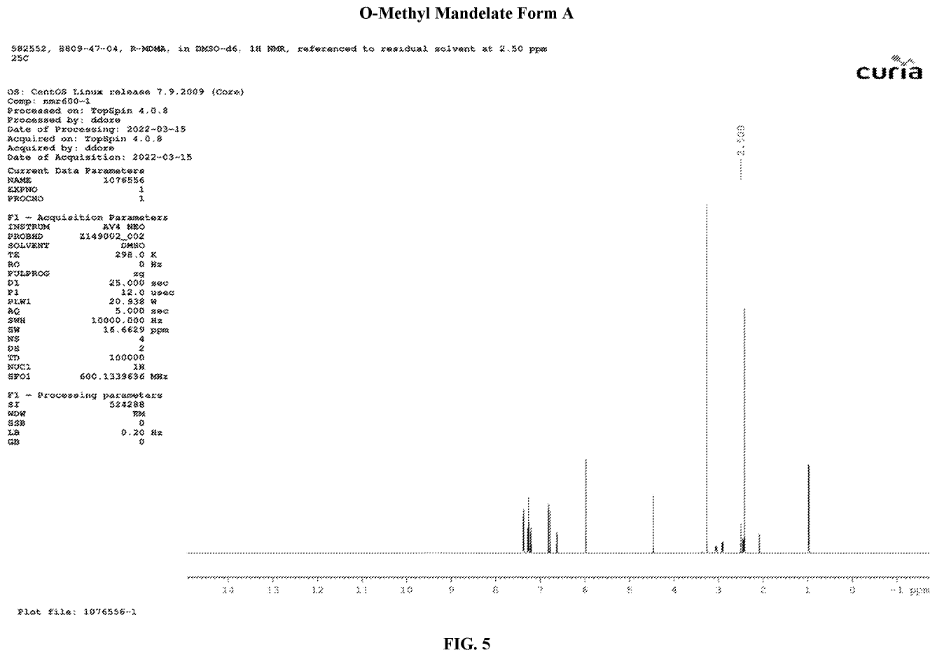

The ¹H NMR spectra of O-Methyl Mandelate Form A is shown in FIG. 5.

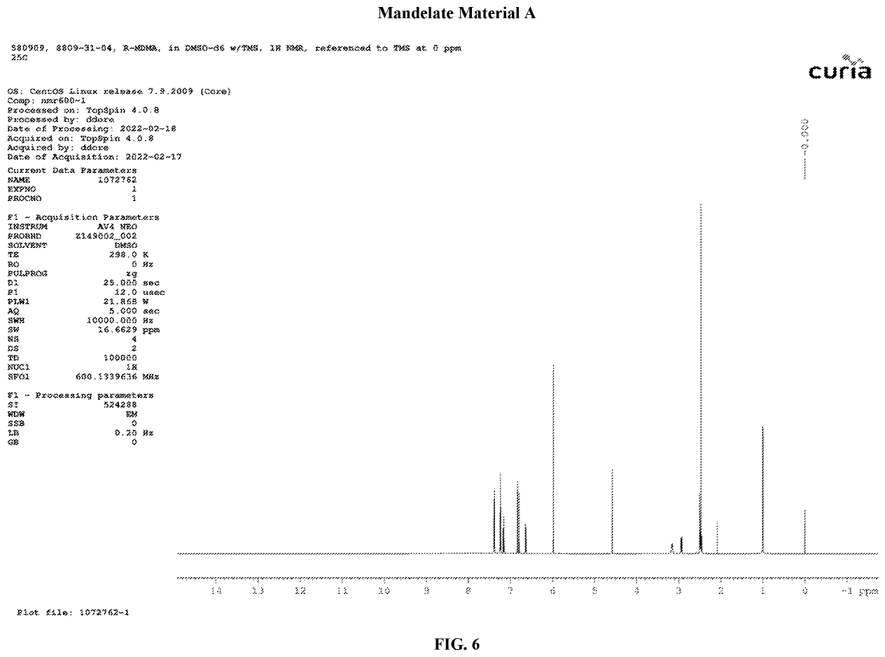

The ¹H NMR spectra of Mandelate Material A is shown in FIG. 6.

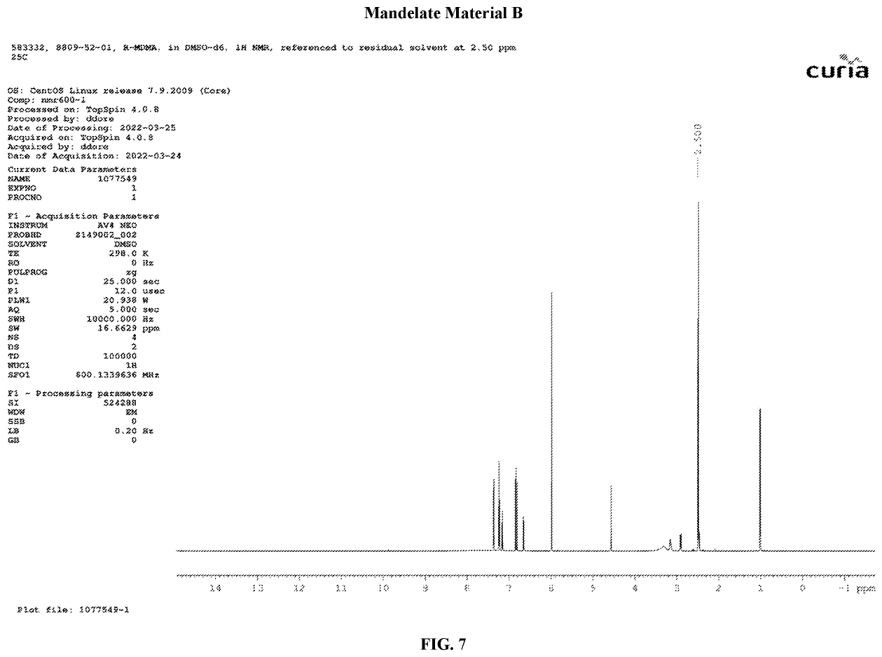

The ¹H NMR spectra of Mandelate Form B is shown in FIG. 7.

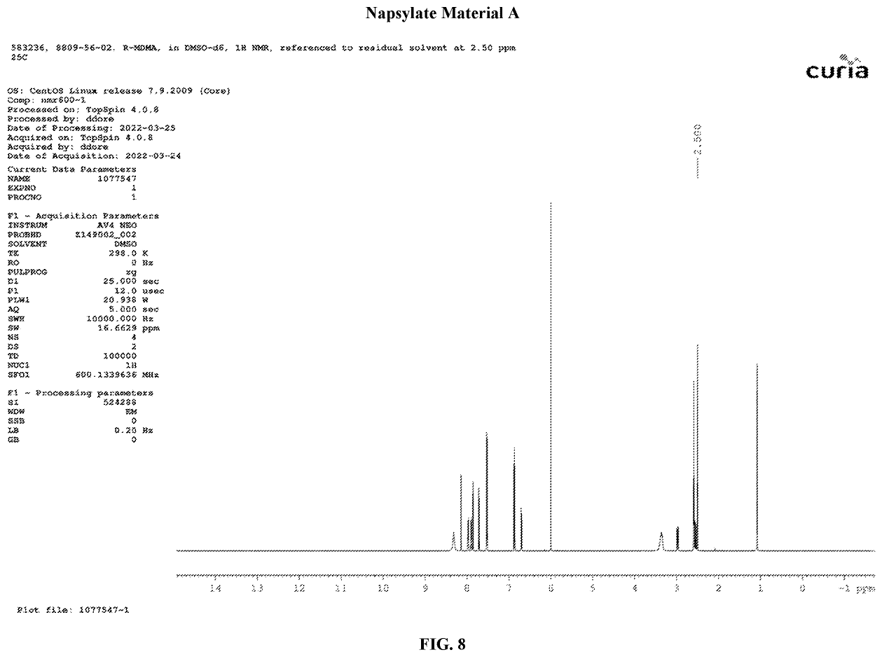

The ¹H NMR spectra of Napsylate Material A is shown in FIG. 8.

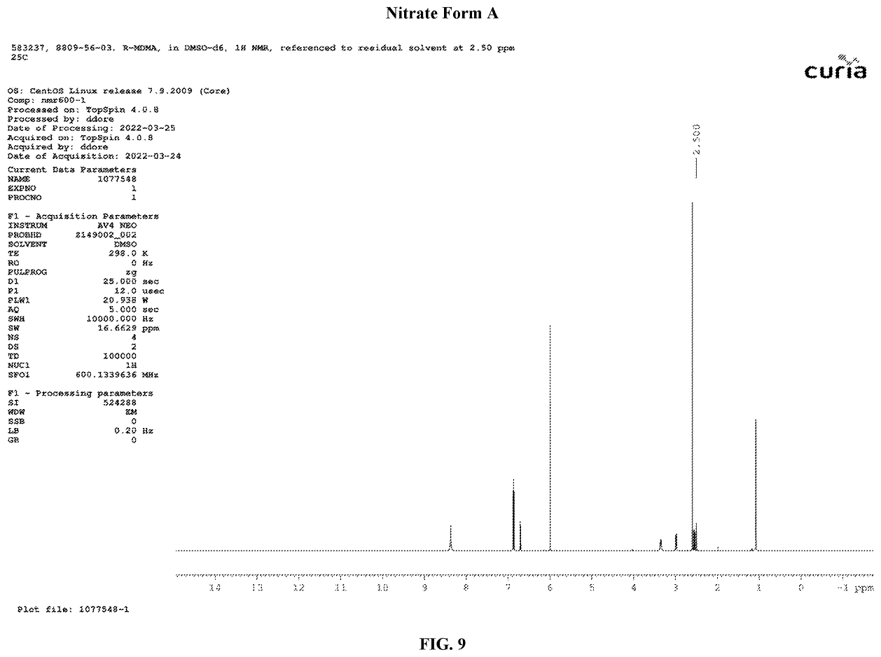

The ¹H NMR spectra of Nitrate Form A is shown in FIG. 9.

The ¹H-NMR results for the five salts are summarized in Table 11 below.

TABLE 11

| R-MDMA Salt | ¹H-NMR observations |
|---|---|
| Mandelate Material A | Consistent with 1:1 MDMA mandelate |
| | salt, negligible acetone |
| Mandelate Form B | Consistent with 1:1 MDMA mandelate salt |
| O-Methyl Mandelate Form A | Consistent with 1:1 MDMA O-methyl |
| | mandelate salt, negligible acetone |
| Napsylate Material A | Consistent with 1:1 MDMA napsylate salt, |
| | negligible EtOAc |
| Nitrate Form A | Consistent with 1:1 MDMA nitrate salt, |
| | negligible EtOAc |

### Thermal Behavior Experiments

### Differential Scanning Calorimetry (DSC):

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, the weight was accurately recorded, and the sample was inserted into the DSC cell. A weighed aluminum pan configured as the sample pan was placed on the reference side of the cell. The pan lid was pierced prior to sample analysis. The samples were analyzed from −25° C. to 250° C. at 10° C./minute unless otherwise stated.

### Thermogravimetric and Differential Scanning Calorimetry (TGA or TGA/DSC):

Thermogravimetric analyses were performed using a Mettler-Toledo TGA/DSC3+ analyzer. Temperature and enthalpy adjustments were performed using indium, tin, zinc, and phenyl salicylate, and then verified with indium. The balance was verified with calcium oxalate. The sample was placed in an aluminum pan. The pan was hermetically sealed, the lid pierced, and the pan was then inserted into the TG furnace. A weighed aluminum pan configured as the sample pan was placed on the reference platform. The furnace was heated under nitrogen. Samples were analyzed from 25° C. to 350° C. at 10° C./min. unless otherwise stated.

### Mandelate Material A:

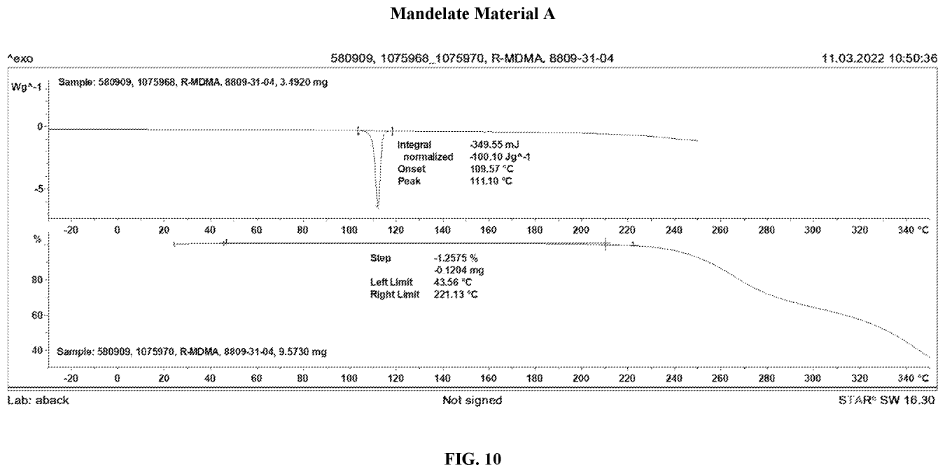

DSC and TGA thermograms of Mandelate Material A were collected at a heating rate of 10° C./min and are shown in FIG. 10. A melting endotherm is observed by DSC at 111° C. with an onset at 110° C. A weight loss of 1.3% is observed between 44-221° C. Decomposition, evidenced by a steep drop in the TGA thermogram, begins above approximately 230° C.

### Mandelate Form B:

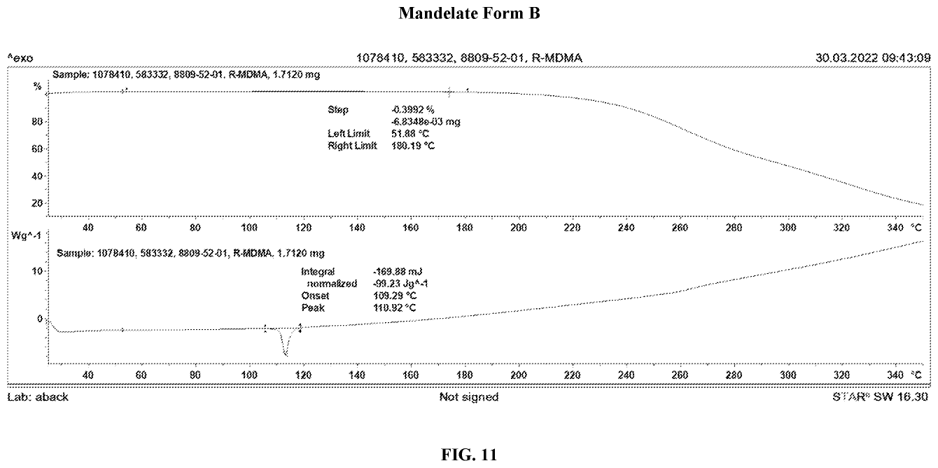

DSC and TGA thermograms of Mandelate Form B were collected at a heating rate of 10° C./min and are shown in FIG. 11. A melting endotherm is observed by DSC at 111° C. with an onset at 109° C. A weight loss of 0.4% is observed between 52-180° C. Decomposition, evidenced by a steep drop in the TGA thermogram, begins above approximately 230° C.

### O-Methyl Mandelate Form A:

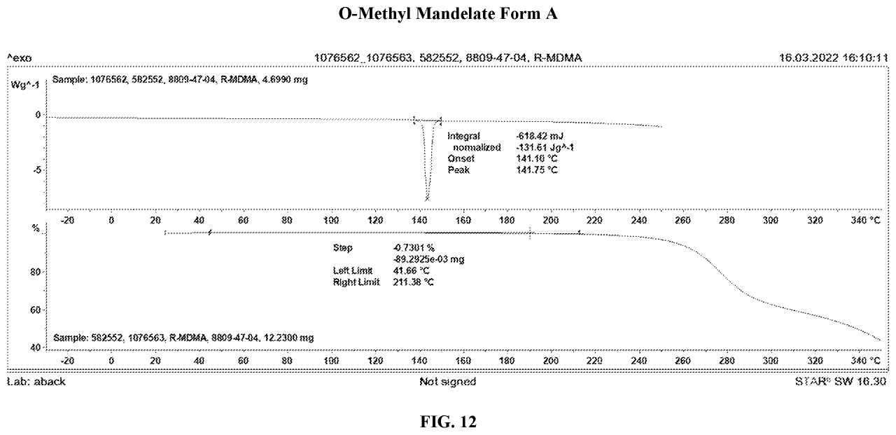

DSC and TGA thermograms of O-Methyl Mandelate Form A were collected at a heating rate of 10° C./min and are shown in FIG. 12. A melting endotherm is observed by DSC at 142° C. with an onset at 141° C. A weight loss of 0.7% is observed between 42-211° C. Decomposition, evidenced by a steep drop in the TGA thermogram, begins above approximately 250° C.

### Napsylate Material A:

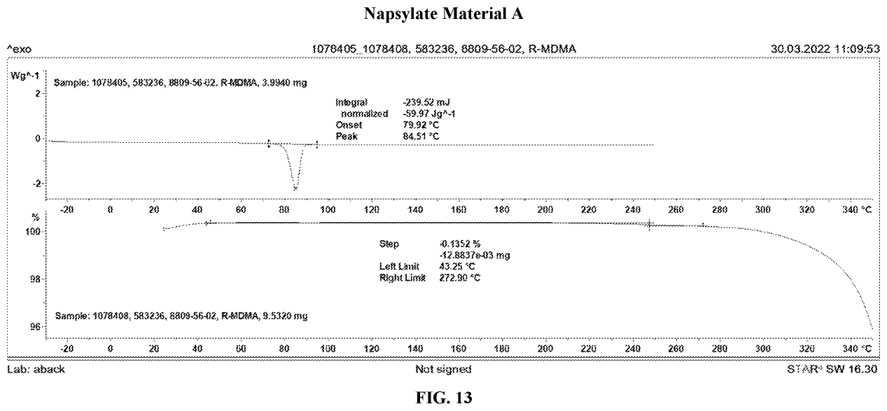

DSC and TGA thermograms of Napsylate Material A were collected at a heating rate of 10° C./min and are shown in FIG. 13. A melting endotherm is observed by DSC at 85° C. with an onset at 80° C. A weight loss of 0.1% is observed between 43-273° C. Decomposition, evidenced by a steep drop in the TGA thermogram, begins above approximately 300° C.

### Nitrate Form A:

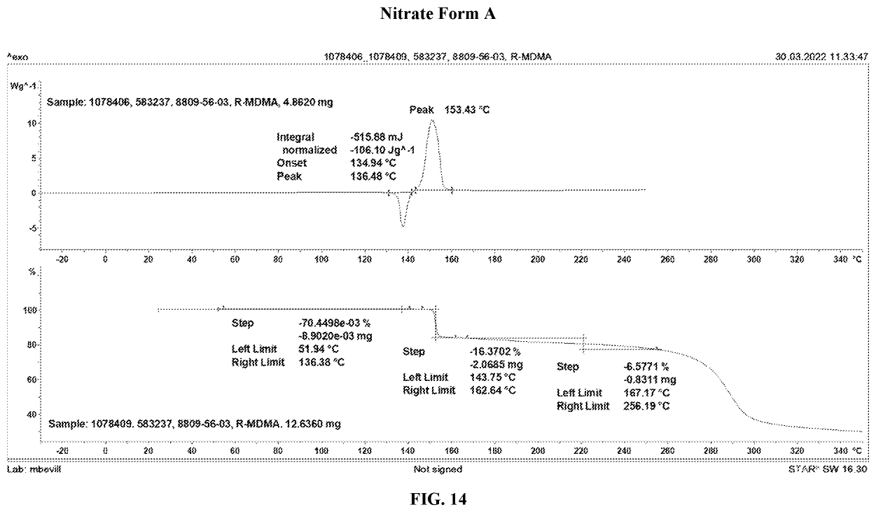

DSC and TGA thermograms of Nitrate Form A were collected at a heating rate of 10° C./min as shown in FIG. 14. A melting endotherm is observed by DSC at 136° C. with an onset at 135° C. A exothermic peak is observed at 153° C. A weight loss of 0.1% is observed between 52-136° C. A weight loss of 16.4% is observed between 144-163° C. A weight loss of 6.6% is observed between 167-256° C. Decomposition, evidenced by a steep drop in the TGA thermogram, begins above approximately 270° C.

The DSC and TGA observations for the five salts are summarized in Table 12 below.

TABLE 12

| R-MDMA salt | DSC observations | TGA observations |
|---|---|---|
| Mandelate Material A | Endothermic peak at 111° C. | 1.3% weight loss 44-221° C. |
| | (onset 110° C.) | |
| Mandelate Form B | Endothermic peak at 111° C. | 0.4% weight loss between |
| | (onset 109° C.) | 52-180° C. |
| O-Methyl Mandelate | Endothermic peak at 142° C. | 0.7% weight loss 42-211° C. |
| Form A | (onset 141° C.) | |
| Napsylate Material A | Endothermic peak at 85° C. | 0.1% weight loss 42-273° C. |
| | (onset 80° C.) | |
| Nitrate Form A | Endothermic peak at 136° C. | 0.1% weight loss 52-136° C. |
| | (onset 135° C.), Exothermic | 16.4% weight loss 144-163° C. |
| | Peak at 153° C. | (eq. 0.6 mol nitric) |
| | | 6.6% weight loss 167-256° C. |
| | | (total wt. loss 144-256° C. eq. |
| | | to 1 mol nitric acid) |

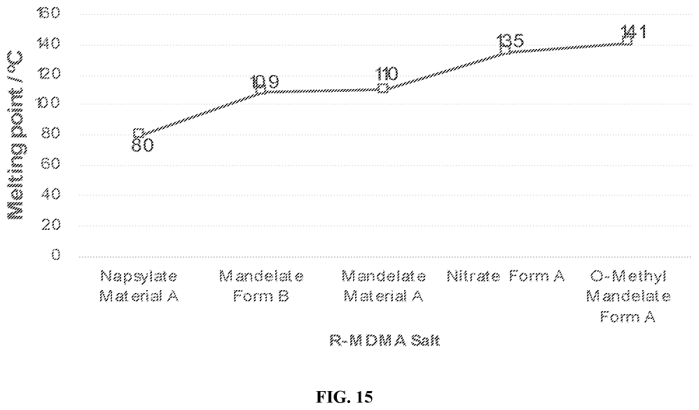

FIG. 15 shows a plot of the melting point temperature of the five R-MDMA salts in Table 12 as observed by DSC.

Napsylate Material A was not confirmed to consist of a single phase, exhibited the lowest melting point, and was obtained in low yields. Therefore, Napsylate Material A was not characterized further.

### Physical Stability Testing

The remaining salts (Mandelate Material A, Mandelate Form B, O-Methyl Mandelate Form A, and Nitrate Form A) were further evaluated by stressing the solids at elevated relative humidity (RH) to monitor for signs of deliquescence or form change by XRPD. None of the stressed materials exhibited signs of deliquescence and O-Methyl Mandelate Form A and Nitrate Form A both remained physically stable upon stressing at ˜90% RH for 8 days and 13 days, respectively. Mandelate Material A appeared to convert to Mandelate Form B upon stressing at ˜90% RH for 9 days. However, reanalysis of the original sample of Mandelate Material A by XRPD after storage at room temperature for 68 days showed conversion of Mandelate Material A to Mandelate Form B. Therefore, the form conversion was not directly attributed to the relative humidity conditions. Mandelate Form B remained physically stable upon stressing at ˜90% RH for 9 days.

### Aqueous Solubility Experiments

The approximate aqueous solubility of each salt was measured by aliquot addition is shown below in Table 13. Both forms of the mandelate salt, Mandelate Material A and Mandelate Form B, were evaluated.

TABLE 13

| | Solubility | |
|---|---|---|
| Salt | (mg/mL) | Observations |
| Mandelate Form B | >82 | clear solution |
| O-Methyl Mandelate Form A | 26 | clear solution |
| Mandelate Material A | 6 | clear solution |
| Nitrate Form A | 3 | clear solution |

Mandelate Form B exhibited the highest solubility of the salts studied with a solubility of >82 mg/mL. O-Methyl Mandelate Form A exhibited the second highest solubility of 26 mg/mL. Limited aqueous solubility was noted for Mandelate Material A of 6 mg/mL and Nitrate Form A of 3 mg/mL. The small sample sizes, aliquot volumes, and kinetic nature of the solubility measurements contribute a degree of uncertainty to the reported values.

### Dynamic Vapor Sorption (DVS) Experiments

Evaluation of the characterization data above, RH stressing results, and approximate aqueous solubility values resulted in the selection of O-Methyl Mandelate Form A as the top salt candidate. O-Methyl Mandelate Form A was additionally characterized by DVS.

DVS data was collected on a Surface Measurement System DVS Intrinsic instrument. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data was not corrected for the initial moisture content of the samples.

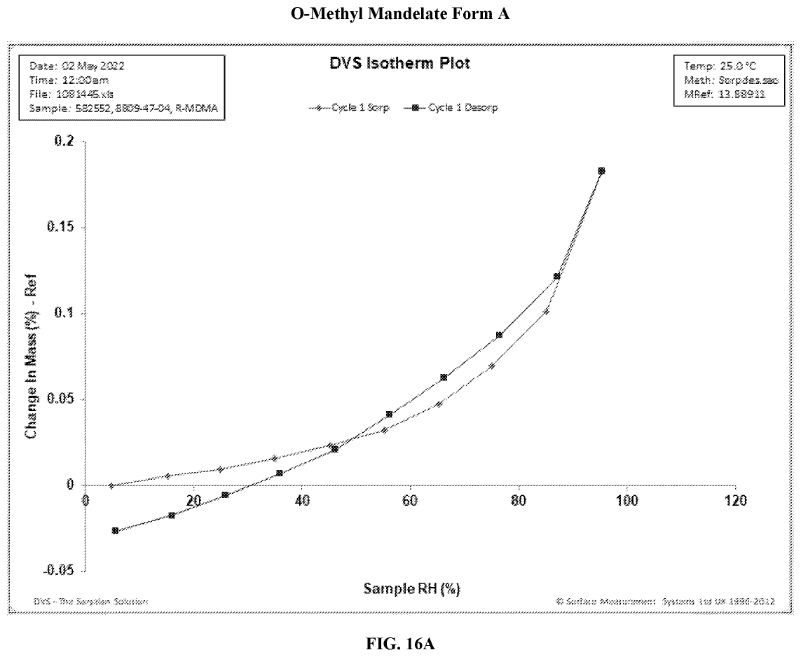

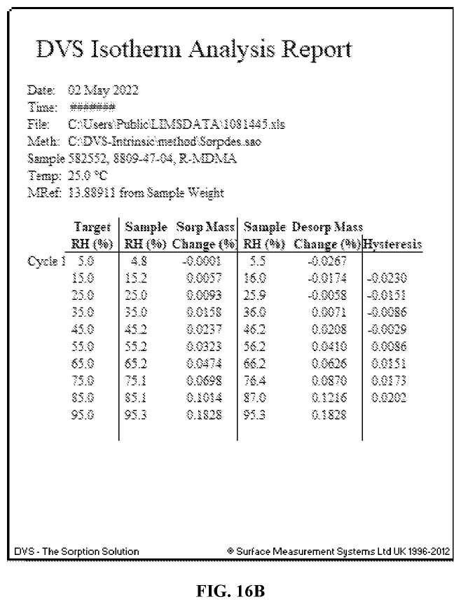

FIG. 16A shows the DVS isotherm and FIG. 16B shows the tabled data of the DVS experiment of O-Methyl mandelate Form A. The isotherm presented in FIG. 16A shows low hygroscopicity of O-Methyl Mandelate Form A. The salt accumulated 0.18% mass water vapor between 5% and 95% RH, and all mass gained in sorption was lost on desorption with minimal hysteresis. The solid form remained unchanged, evaluated by a XRPD of the post-DVS solids of O-Methyl Mandelate Form A.

### CONCLUSION

Approximately 23 salt screen experiments for R-MDMA free base were conducted with nine different acids (Table 1). R-MDMA used in this study was synthesized using the method described in U.S. patent application Ser. No. 17/901,504, filed Sep. 21, 2022 and entitled “SYNTHESIS OF MDMA OR ITS OPTICALLY ACTIVE (R)- OR (S)-MDMA ISOMERS”, which is hereby incorporated by reference in its entirety. Confirmed or suspected salts produced from the screen include Mandelate Material A, Mandelate Form B, O-Methyl Mandelate Form A, Napsylate Form A, Nitrate Form A, Tartaric Material A+tartaric acid, and Tartaric Material B+Tartaric Material A+tartaric acid. Table 14 below shows the summarized characterization of these seven salts below.

TABLE 14

| Material/ | | |
|---|---|---|
| Form | Technique | Results |
| Mandelate | XRPD | Mandelate Material A; could not be indexed |
| Material A | ¹H NMR | Consistent with 1:1 MDMA Mandelate salt, |
| | | negligible acetone |
| | DSC | Endothermic peak at 111° C. (onset 110° C.) |
| | TGA | 1.3% weight loss 44-221° C. |
| | XRPD | Mandelate Form B (68 days post initial analysis) |
| Mandelate | XRPD | Mandelate Form B; successfully indexed, consistent |
| Form B | | with 1:1 MDMA Mandelate salt |
| | ¹H NMR | Consistent with 1:1 MDMA mandelate salt |
| | DSC/TGA | Endothermic peak at 111° C. (onset 109° C.) |
| | | 0.4% weight loss between 52-180° C. |
| O-methyl | XRPD | O-Methyl Mandelate Form A; successfully indexed, |
| Mandelate | | consistent with a 1:1 MDMA O-methyl mandelate |
| Form A | ¹H NMR | Consistent with 1:1 MDMA O-Methyl Mandelate |
| | | salt, negligible acetone |
| | DSC | Endothermic peak at 142° C. (onset 141° C.) |
| | TGA | 0.7% weight loss 42-211° C. |
| | DVS | 0.18% weight gain 5-95%, 0.21% weight loss |
| | | 95-6% RH |
| | post-DVS | O-Methyl Mandelate Form A |
| | XRPD | |
| Napsylate | XRPD | Napsylate Material A; could not be indexed |
| Material A | ¹H NMR | Consistent with 1:1 MDMA Napsylate salt, |
| | | negligible EtOAc |
| | DSC | Endothermic peak at 85° C. (onset 80° C.) |
| | TGA | 0.1% weight loss 43-273° C. |
| Nitrate | XRPD | Nitrate Form A; successfully indexed, consistent |
| Form A | | with 1:1 MDMA nitric salt |
| | ¹H NMR | Consistent with 1:1 MDMA Nitrate salt, negligible |
| | | EtOAc |
| | DSC | Endothermic at 136° C. (onset 135° C.), Exothermic |
| | | peak at 153° C. |
| | TGA | 0.1% weight loss 52-136° C. |
| | | 16.4% weight loss 144-163° C. (eq. 0.6 mol nitric) |
| | | 6.6% weight loss 167-256° C. |
| | | (total wt. loss 144-256° C. eq. to 1 mol nitric acid) |
| Tartaric | XRPD | Tartaric Material A + tartaric acid; could not be indexed |
| Material A + | | |
| tartaric acid | | |
| Tartaric | XRPD | Tartaric Material B + Tartaric Materiial A + tartaric |
| Material B + | | acid; could not be indexed |
| Tartaric | | |
| Material A + | | |
| tartaric acid | | |

For an API, an ideal R-MDMA salt candidate consists of an anhydrous/unsolvated stoichiometric salt confirmed to consist of a single crystalline phase with a melt onset above 125° C. From the data presented in Table 14 above, O-Methyl Mandelate Form A and Nitrate Form A meet the criteria for an ideal API. On the contrary, the remaining salts exhibited less suitable characteristics, making them less suitable as an API. Mandelate Form B consists of an anhydrous/unsolvated 1:1 salt in a single phase but exhibits a relatively low melt onset (109° C.). The XRPD pattern for Mandelate Material A was unable to be indexed, the material exhibited a relatively low melt onset (110° C.), and the Mandelate Material A converted to Mandelate Form B upon storage at ambient conditions. Napsylate Material A exhibited the lowest melting point, was not confirmed to consist of a single phase, and was obtained in low yields. Napsylate Material A was not characterized further. The two potential tartrate materials were observed only in mixtures with unreacted tartaric acid and were not characterized further.

O-Methyl Mandelate Form A comprises of an anhydrous/unsolvated mono-salt that was confirmed to consist of a single phase and exhibited the following desirable properties: a melt onset at 141° C., approximate aqueous solubility of 26 mg/mL, low kinetic hygroscopicity, and physical stability at 90% RH. Based on this study, O-Methyl Mandelate Form A was identified as a top candidate with suitable properties for pharmaceutical development.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. (R)-3,4-methylenedioxymethamphetamine ((R)-MDMA) O-methyl mandelate.

2. The (R)-MDMA O-methyl mandelate of claim 1, wherein the (R)-MDMA O-methyl mandelate is crystalline.

3. The crystalline (R)-MDMA O-methyl mandelate of claim 2, characterized by X-ray powder diffraction (XRPD) peaks at 16.0±0.2, 16.8±0.2, and 18.2±0.2°2θ.

4. The crystalline (R)-MDMA O-methyl mandelate of claim 3, further characterized by XRPD peaks at 2.5±0.2, 16.2±0.2, and 23.0±0.2°2θ.

5. The crystalline (R)-MDMA O-methyl mandelate of claim 2, which exhibits a Differential Scanning Calorimetry (DSC) thermogram comprising an endotherm peak with an onset at about 141° C.

6. The crystalline (R)-MDMA O-methyl mandelate of claim 2, which exhibits a DSC thermogram comprising an endothermic peak at 142° C.±5° C.

7. The crystalline (R)-MDMA O-methyl mandelate of claim 2, which exhibits 0.7±0.10% weight loss between 42±5 to 211±5° C. as measured by thermogravimetric (TGA) analysis.

8. The crystalline (R)-MDMA O-methyl mandelate of claim 2, which has a melting point of 141±5° C.

9. The crystalline (R)-MDMA O-methyl mandelate of claim 2, which exhibits 0.18±0.05% weight gain from 5-95% relative humidity (RH) and a 0.21±0.05% weight loss from 95-6% RH as measured by Dynamic Vapor Sorption (DVS) under a nitrogen purge.

10. The crystalline (R)-MDMA O-methyl mandelate of claim 2, which is stable after storage for 8 days at 90% relative humidity (RH) at about 23° C.

11. The crystalline (R)-MDMA O-methyl mandelate of claim 2, which exhibits an aqueous solubility of 26±5 mg/mL.

12. The crystalline (R)-MDMA O-methyl mandelate of claim 2, which exhibits a primitive monoclinic system with cell parameters of a=6.26±0.05 Å, b=51.09±0.05 Å, c=6.41±0.05 Å, α=90±1°, β=113.4±1°, γ=90±1°, and a cell volume of 1,882±5 Å³/cell.

13. A composition comprising the (R)-MDMA O-methyl mandelate of claim 1 and a pharmaceutically acceptable excipient.

14. A composition comprising the crystalline (R)-MDMA O-methyl mandelate of claim 2 and a pharmaceutically acceptable excipient.

15. An oral dosage form comprising the composition of claim 13.

16. An oral dosage form comprising the composition of claim 14.
